(19) United States
(12) Patent Application Publication
(10) Pub. No.: US 2014/0107476 A1
(43) Pub. Date: Apr. 17, 2014

(54) SPATIAL SAMPLING IMPROVEMENT FOR LIST-MODE PET ACQUISITION USING PLANNED TABLE/GANTRY MOVEMENT

(71) Applicants: Chi-Hua Tung, Aurora, OH (US); Bin Zhang, Cleveland, OH (US); Changhong Dai, Willoughby Hills, OH (US)

(72) Inventors: Chi-Hua Tung, Aurora, OH (US); Bin Zhang, Cleveland, OH (US); Changhong Dai, Willoughby Hills, OH (US)

(73) Assignee: KONINKLIJKE PHILIPS N.V., EINDHOVEN (NL)

(21) Appl. No.: 14/124,351

(22) PCT Filed: Jun. 1, 2012
(86) PCT No.: PCT/IB2012/052762

§ 371 Date: Dec. 6, 2013

Related U.S. Application Data
(60) Provisional application No. 61/497,548, filed on Jun. 16, 2011.

(51) Int. Cl. A61B 6/03

(52) U.S. Cl. CPC A61B 6/037
USPC 600/425

(57) ABSTRACT

A PET apparatus includes a detector array including individual detectors which receive radiation events from an imaging region. A movement controller controls at least one of relative longitudinal movement between a subject support and the detector array and circumferential movement between the detector array and the subject. A time stamp processor assigns a time stamp to each received radiation event. A list mode event storage buffer stores time stamped events. An event verification processor screens for coincidentally received radiation events, locations at which each pair of corresponding coincidentally received events defining a line of response. A reconstruction processor reconstructs valid events into an image representation of the imaging region.

The present application relates to the diagnostic imaging arts. It finds particular application in utilizing planned table and/or gantry movement to achieve improved spatial sampling for list-mode PET acquisition. It is to be understood, however, that it also finds application in other devices, and is not necessarily limited to the aforementioned application.

Nuclear imaging devices, e.g. positron emission tomography (PET) scanners, reconstruct images from lines of response (LORs) in a field of view (FOV). An image value for a voxel is generated by summing a contribution of each LOR which intersects the voxel. In list-mode acquisition, events are recorded one-by-one into a list file and are regarded as an independent data points used in the reconstruction. In current clinical PET imaging, PET data acquisition is commonly done at fixed table positions. Both the scanner and table remain static during the acquisition resulting in fixed detector geometries and unchanged spatial data sampling over the FOV.

Due to the limited PET FOV, the image reconstructed at one position may not be able to cover the entire imaging object, e.g. in whole body imaging. Therefore, acquisitions at multiple table positions are acquired to form the whole image of the object. Due the limited crystal size of the PET scanner, PET acquisition always has limited sampling of the FOV (in both axial and transversal directions), which limits the PET image resolution. As patient table and scanner gantry commonly remains static during the data acquisition, where an event can be acquired is fully dependent upon crystal locations and scanner geometries.

The present application provides a new and improved system and method which overcomes the above-referenced problems and others.

In accordance with one aspect, a PET apparatus is provided. The PET apparatus includes a detector array including individual detectors which receive radiation events from an imaging region. A movement controller controls at least one of relative longitudinal movement between a subject support and the detector array and circumferential movement between the detector array and the subject. A time stamp processor assigns a time stamp to each received radiation event. A list mode event storage buffer stores time stamped events. An event verification processor screens for coincidentally received radiation events, locations at which each pair of corresponding coincidentally received events defining a line of response. A reconstruction processor reconstructs valid events into an image representation of the imaging region.

In accordance with another aspect, a method is provided. The method includes receiving radiation events from an imaging region, controlling at least one of relative longitudinal movement between a subject support and the detector array and circumferential movement between the detector array and the subject, assigning a time stamp to each received radiation event, storing valid time stamped events, screening for coincidentally received radiation events, defining a line of response at each pair of corresponding coincidentally received events; and reconstructing valid events into an image representation of the imaging region.

In accordance with another aspect, A PET imaging apparatus is provided. The PET imaging apparatus includes a detector array which surrounds an imaging region. One or more motors move the detector array at least one of circumferentially and longitudinally. One or more processor programmed to identify pairs of radiation events coincidently received by a pair of detectors of the array, define a line of response based on at least one of longitudinal and circumferential location of the detectors which receive a corresponding coincident pair of events, and reconstruct the line of responses into an image.

One advantage resides in improved spatial sampling of PET data.

Another advantage resides in improved image resolution.

Another advantage resides in a larger effective field of view.

Still further advantages of the present invention will be appreciated to those of ordinary skill in the art upon reading and understand the following detailed description.

The invention may take form in various components and arrangements of components, and in various steps and arrangements of steps. The drawings are only for purposes of illustrating the preferred embodiments and are not to be construed as limiting the invention.

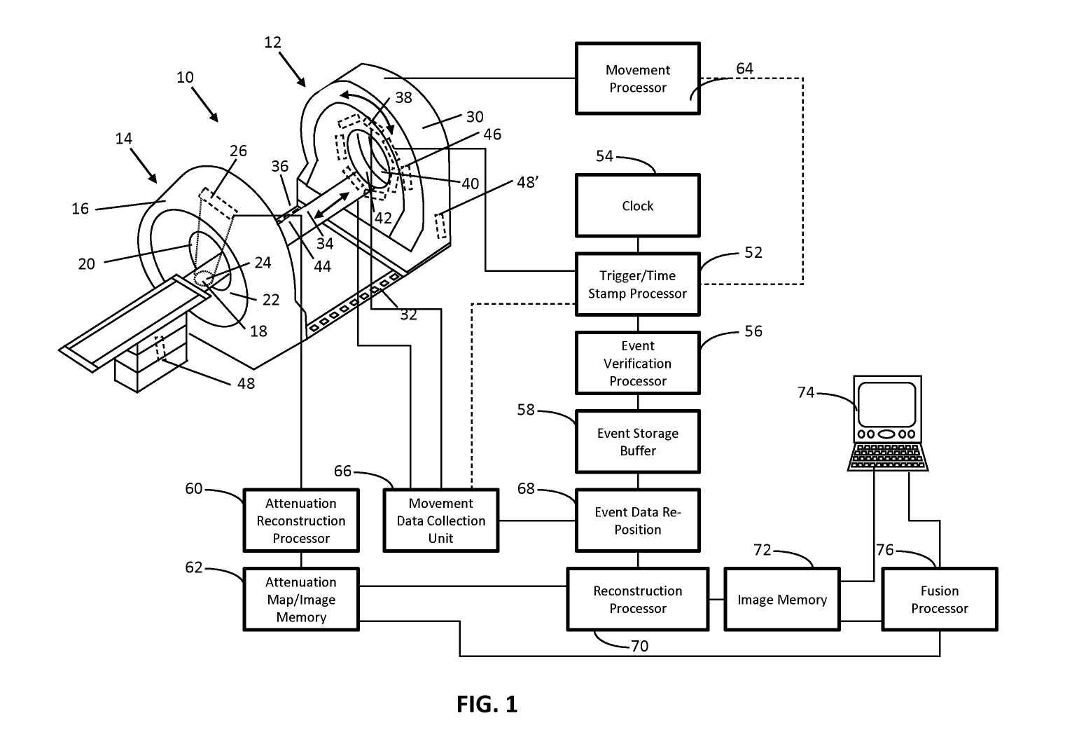

FIG. 1 is a diagrammatic illustration of an imaging system in accordance with the present application.

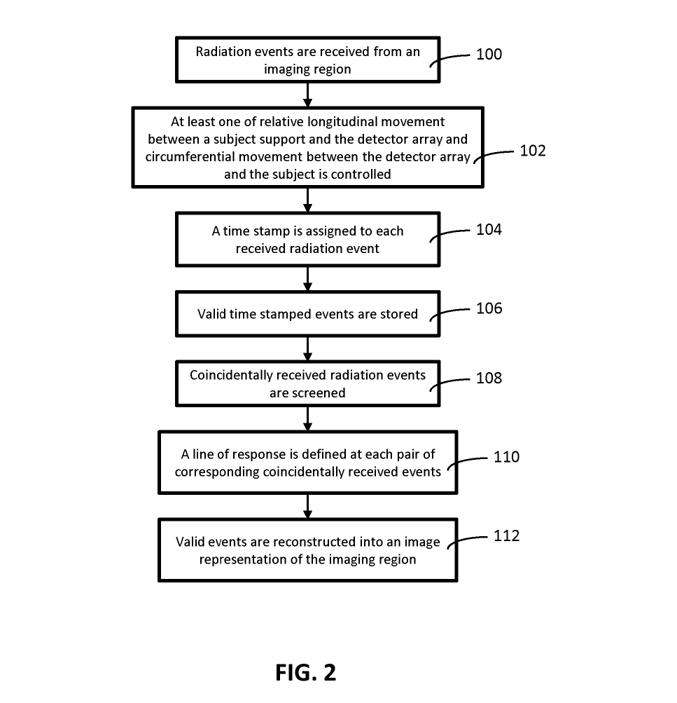

FIG. 2 is a flowchart illustration of a method of image processing in accordance with the present application.

FIG. 1 illustrates a multi-modality system 10 which implements a workflow that utilizes planned table/gantry movement to acquire list-mode events at different spatial sampling locations to improve spatial sampling of PET data to image resolution. The workflow, described in detail below, utilizes planned table/gantry movement during data acquisition. By this approach, each crystal can cover different spatial locations during the scan which results in finer PET acquisition sampling in the field of view (FOV). With accurate knowledge of the table and gantry movement and timing, the line of response (LOR) of each list-mode event can be calculated based on the timing the event and the planned movement. The movement can be in a short distance, such as in single or multiple half-crystal length. Table movement will mainly benefit sampling in axial direction while gantry rotation-like movement can improve sampling in transversal direction. In addition, the table movement can also help increasing scanning axial FOV and reducing truncation effect in data corrections.

With reference to FIG. 1, a multi-modality system 10 includes a first imaging system, e.g. a functional modality, preferably, a nuclear imaging system 12, and a second imaging system, e.g. an anatomical modality, such as a computed tomography (CT) scanner 14, magnetic resonance (MR) scanner, a C-arm x-ray scanner, and the like. The CT scanner 14 includes a non-gantry 16. An x-ray tube 18 is mounted to a gantry 20. A bore 22 defines an examination region 24 of the CT scanner 14. An array of radiation detectors 26 is disposed on the gantry 20 to receive radiation from the x-ray tube 18 after the x-rays transverse the examination region 24. Alternatively, the array of detectors 26 may be positioned on the non-gantry 16. Of course, magnetic resonance and other imaging modalities are also contemplated.

The functional or nuclear imaging system 12, in the illustrated embodiment, includes a positron emission tomography (PET) scanner 30 which may be mounted on tracks 32 to facilitate patient access. Of course, SPECT, CT, nuclear medicine imaging, and other imaging modalities are also contemplated. The tracks 32 extend in parallel to a longitudinal axis of a subject support or table 34, thus enabling the CT scanner 14 and PET scanner 12 to form a closed system. A motor and drive 36, is provided to move the PET scanner 12 in and out of the closed position and/or to move the patient and the scanner relative to each other. Detectors 38 are arranged around a gantry 40 which defines an examination region 42. The gantry is mounted to oscillate or rotate 44 over an arc that is at least a center to center spacing between radically adjacent detector elements. A rotational motor and drive 46 or the like, provide the oscillator or rotational movement of the detectors relative to the patient. When the detectors move continuously, the detectors are sequentially disposed over a continuum of detector locations. Alternatively, the detectors can be stepped in increments. For uniformity, in one embodiment, the detectors spend a like amount of time in each position. A longitudinal motor and drive 48, 48′ or the like, provides relative longitudinal movement between the subject support 34 and the PET detectors. In one embodiment, the longitudinal motors and drive 48 moves the subject support. In another embodiment, the longitudinal motor and drive 48; moves the PET gantry, hence the detectors. Combined CT and PET systems in a single, shared close gantry with a common examination region are also contemplated.

With continued reference to FIG. 1, the subject support 34, which carries a subject, is positioned in the examination region 24 of the CT scanner 14. The CT scanner 14 generates radiation attenuated data which is then used by an attenuation reconstruction processor 60 to reconstruct the radiation attenuated data into an attenuation map or anatomical attenuation image that is stored in an attenuation memory 62. A high resolution CT image can be used as the attenuation map. Alternatively, the attenuation map can have relatively low spatial and contrast resolution. The patient support 34 moves the subject into the PET scanner 12 in a position that is geometrically and mechanically predicated as being the same as the imaged position in the CT imaging region 24. To generate an image over a longitudinally elongated region, the patient is positioned at a common starting position in the CT and PET scanners and translated over a corresponding anatomical region. Due to different scanning speeds for the CT and PET scanners, the longitudinal displacement speeds may be different. Before the PET scan commences, a subject is injected with a radiopharmaceutical. In PET scanning, a pair of gamma rays is produced by a positron annihilation event in the examination region 42 and travel in opposite directions. When the gamma ray strikes the detectors 38, the location of the struck detector element and the strike time are recorded. A triggering and time stamp processor 52 monitors each detector 38 for an energy spike, e.g., integrated area under the pulse, characteristic of the energy of the gamma rays generated by the radiopharmaceutical. The triggering and time stamp processor 52 checks a clock 54 and stamps each detected gamma ray event with a time of leading edge receipt and, in a time of flight scanner, a time of flight (TOF). In PET imaging, the time stamp, energy estimate, and a location of the detector are first used by an event verification processor 56 to determine whether there is a coincident event. Accepted pairs of coincident events define lines of response (LORs). Once an event pair is verified by the event verification processor 56, the LOR is passed to an event storage buffer 58 with their time stamps and end point detectors locations are stored in the event storage buffer 58 as event data.

The subject support 34 and/or the PET gantry are continuously or stepwise moved relative to each other to generate list-mode PET data sets that contain events associated with their corresponding location information of the detectors that detect the paired photons. This allows each detector to cover a continuum of longitudinal spatial locations during the scan which results in finer PET acquisition sampling in the longitudinal or z direction. Stepping in short longitudinal increments, e.g. smaller than the longitudinal detector spacing, is also contemplated. The detectors are also moved circumferentially continuously or in analogous small steps. The longitudinal and rotational movement speeds can be different. To accomplish this, the system is configured with a movement processor 64 which controls the relative movement of the subject support 34 and/or the gantry 40. The movement processor 64 plans the timing and longitudinal and rotational movement pattern of the subject support 34 and/or the gantry 40 including the movement distance, speed, direction, and the like. It should be appreciated that during data acquisition both the subject support 34 and the gantry 40 can move, the subject support 34 can move by itself, or the gantry 40 can move circumferentially by itself. In one embodiment, the movement processor 64 provides the current intended longitudinal circumferential location, e.g. offset from a starting or reference location, of the detector to the trigger/time stamp processor 52 which adjusts the location of the detector detecting each event accordingly. In another embodiment, a movement data collection unit 66 measures the longitudinal location of the subject support 34 and/or the gantry and the circumferential location of the detectors. Actual movement data including the longitudinal location, circumferential location, and the like are measured by one or more sensors 66L, 66C which sense the relative longitudinal location of the subject support 34 and/or the gantry 40 and the circumferential location of the detectors respectively. In one embodiment, the movement data is utilized by an event data reposition processor 68 which adjusts or corrects the LOR trajectory of each list-mode event, e.g. shifts the end or detection points of each LOR, based on the scanner geometry and the movement information.

A reconstruction processor 70 reconstructs the location adjusted or corrected LORs into an image representation of the subject using the attenuation map or image for attenuation correction. In one embodiment, a list-mode reconstruction algorithm is used. The reconstruction processor 70 reconstructs the image representation from the adjusted or corrected LORs by generating an image value for each voxel including the contribution of each adjusted or corrected LOR which intersects the voxel. The voxel can have a shape of a rectangular prism, e.g. a cube, a blob, or the like. The reconstructed image is stored in an image memory 72 and displayed for a user on a display device 74, printed, saved for later use, and the like. In one embodiment, a fusion processor 76 combines the functional, PET image with the anatomical, attenuation image.

In one embodiment, the event data is collected in a list-mode format. Recording the relevant properties (detector coordinates, time stamp, etc.) of each detected event in a list has become known as list-mode data acquisition and storage. The list-mode format also includes or is adjusted for the movement data for each event data such that each LOR of each list-mode event can be adjusted or corrected based on the scanner geometry and the movement data. This enables the subject support 34 and/or the gantry 40 to be moved continuously, in small steps, or the like during data acquisition. By collecting the data with the positional information that is collected on a finer grid than the traditional spacing between the detector elements the resolution of the system and the resultant PET image can be improved.

The triggering processor 52, event verification processor 56, attenuation reconstruction processor 60, reconstruction processor 70, and the movement processor 64 include a common or different processor, for example a microprocessor or other software controlled device configured to execute image reconstruction software for performing the operations described in further detail below. Typically, the image reconstruction software is carried on tangible memory or a computer readable medium for execution by the processor. Types of computer readable media include memory such as a hard disk drive, CD-ROM, DVD-ROM and the like. Other implementations of the processor are also contemplated. Display controllers, Application Specific Integrated Circuits (ASICs), FPGAs, and microcontrollers are illustrative examples of other types of component which may be implemented to provide functions of the processor. Embodiments may be implemented using software for execution by a processor, hardware, or some combination thereof.

In one embodiment, the movement controller 64 controls the subject support 34 to move continuously along the longitudinal axis of a subject support 34. The movement controller 64 controls the distance, direction, and speed of the subject support 34. The movement of the subject support 34 is continuous but it is also contemplated that the movement is in a series of short steps. Likewise, the speed of the subject support 34 is preferably constant but it is also contemplated that the speed varies of the subject support 34 varies based on the imaging application. For example, the subject support 34 may move at non-continuous speeds in order to gain more detail for certain regions of interest, to compensate for sampling variations at the beginning and end of the longitudinal movement, and the like. It should be appreciated that the movement of the subject supports 34 is controlled such that the count rate is adequate for PET imaging. For example, the movement controller 64 controls the subject support 34 to move at a rate of 9 centimeters per minute which will provides adequate or event counts for image acquisition. The continuous movement further decreases the total time for image acquisition. Because there is no longer a need to move the subject support and/or gantry in a series of short steps, the time for image acquisition is reduced. For example, image acquisition utilizing continuous gantry and/or subject support movement is performed in half the time of traditional image acquisition. Image acquisition time is also reduced by moving the subject support 34 at non-continuous speeds through regions of non-interest. For example, the subject speed may be increased in regions that are not of interest to the clinician to reduce image acquisition time.

The movement controller 64 also controls the gantry 40 to rotate or oscillate in the circumferential direction in either continuously or in a series of short steps. Typically, the rotation is only over an arc which spans about the center-to-center spacing of circumferentially adjacent detectors. As described above, the movement controller 64 controls the distance, direction, and speed of the gantry 40. The movement controller 34 plans the timing and the movement pattern of the subject support 34 and/or the gantry 40 including the moving distance and the moving direction for each PET scanning sequence.

The movement data collection unit 66 measures the actual relative longitudinal locations of the subject support 34 and/or the gantry 40 and the circumferential location of the gantry and generate movement data indicative of such. In one embodiment, the movement data includes the movement distance, direction, speed and the like is used to create a motion model which is used to correct the LORs. In another embodiment, the movement data collection unit 66 also records time stamps with the measure locations of the event data which are used by the event data reposition processor 68 to correlate the location adjustments with the LORs. For example, from the movement data, the event data reposition processor 68 determines the position, time, and displacement of each detector for each list-mode event detected. The location is typically measured as a displacement amount from a reference location. The event data reposition processor 68 utilizes this information to adjust or correct the LOR of each list-mode event based on the scanner and detector geometry. The displacement amounts are then used to physically shift, reorient, or adjust the LORs

FIG. 2 illustrates a method of image processing. In a step 100, radiation events are received from an imaging region. In a step 102, at least one of relative longitudinal movement between a subject support and the detector array and circumferential movement between the detector array and the subject is controlled. In a step 104, a time stamp is assigned to each received radiation event. In a step 106, valid time stamped events are stored. In a step 108, coincidentally received radiation events are screened. In a step 110, a line of response is defined at each pair of corresponding coincidentally received events. In a step 112, valid events are reconstructed into an image representation of the imaging region.

The invention has been described with reference to the preferred embodiments. Modifications and alterations may occur to others upon reading and understanding the preceding detailed description. It is intended that the invention be constructed as including all such modifications and alterations insofar as they come within the scope of the appended claims or the equivalents thereof.

1. A PET apparatus comprising:
a detector array including individual detectors which receives radiation events from an imaging region;
a movement controller which controls at least one of relative longitudinal movement between a subject support and the detector array and relative circumferential movement between the subject support and the detector array;
a time stamp processor which assigns a time stamp to each received radiation event;
a list mode event storage buffer which stores time stamped events;
an event verification processor which screens for coincidentally received radiation events and locations at which each pair of corresponding coincidentally received events defining a line of response.
a reconstruction processor which reconstructs valid events into an image representation of the imaging region.

2. The diagnostic imaging apparatus as set forth in claim 1, wherein the at least one of the relative longitudinal movement between the subject support and the detector array and the relative circumferential movement between the detector array and the subject support is continuous.

3. The diagnostic imaging apparatus as set forth in claim 1, further including:
a event data reposition processor which adjusts corresponding lines of response of the received events based on a current longitudinal and circumferential location of the detectors which receive the radiation events.

4. The diagnostic imaging apparatus as set forth in claim 1, wherein the time stamp processor assigns a current longitudinal and circumferential location of the detectors that received each radiation event.

5. The diagnostic imaging apparatus as set forth in claim 1, further including:
a movement data collection unit which measures a current relative longitudinal location between the subject support and the detector array and a current relative circumferential location between the detector array and the subject support.

6. The diagnostic imaging apparatus as set forth in claim 1, wherein the circumferential movement detector elements of the detector array is sequentially disposed over a continuum of detector locations.

7. The diagnostic imaging apparatus as set forth in claim 1, wherein the movement controller reduces image acquisition time by adjusting one or more scanning parameters.

8. The diagnostic imaging apparatus as set forth in claim 1, wherein the relative circumferential movement is over an arc that is at least a center to center spacing between adjacent detector elements of the detector array.

9. The diagnostic imaging apparatus as set forth in claim 1, wherein one of the relative longitudinal movement between the subject support and the detector array and the relative circumferential movement between the detector array and the subject support is stepped in short longitudinal increments smaller than the center-to-center distance of longitudinal or circumferential adjacent detector elements.

10. A method comprising:
receiving radiation events from an imaging region;
controlling at least one of relative longitudinal movement between a subject support and the detector array and relative circumferential movement between the detector array and the subject support;
assigning a time stamp to each received radiation event;
storing valid time stamped events;
screening for coincidentally received radiation events;
defining a line of response between locations of a pair of detector elements which received the coincidentally received events; and
reconstructing the LORs into an image representation of the imaging region.

11. The method as set forth in claim 10, wherein the at least one of the relative longitudinal movement between the subject support and the detector array and the relative circumferential movement between the detector array and the subject support is continuous.

12. The method as set forth in claim 10, further including:
adjusting corresponding lines of response of the received events based on a current longitudinal and circumferential location of the detectors which receive the radiation events.

13. The method as set forth in claim 10, further including:
with a time stamp processor assigning a time stamp a current longitudinal and circumferential location of the detector which received the radiation event.

14. The method as set forth in claim 10, further including:
measuring one of a current relative longitudinal location of the subject support and the detector array and a current relative circumferential location of the detector array and the subject support.

15. The method as set forth in claim 10, wherein the circumferential movement detector elements of the detector array is sequentially disposed over a continuum of detector locations.

16. The method as set forth in claim 10, further including:
reducing image acquisition time by adjusting one or more scanning parameters.

17. The method as set forth in claim 10, wherein the relative circumferential movement is over an arc that is at least a center to center spacing between adjacent detector elements of the detector array.

18. The method as set forth in claim 10, wherein one of the relative longitudinal movement between the subject support and the detector array and the relative circumferential movement between the detector array and the subject support is stepped in short longitudinal increments smaller than the longitudinal or circumferential adjacent detector elements.

19. A non transitory computer readable medium which carries a computer program which controls one or more processors to perform the method of claim 10.

20. A PET imaging apparatus comprising:
a detector array which surrounds an imaging region;
one or more motors which moves the detector array at least one of circumferentially and longitudinally;
one or more processor programmed to:
identify pairs of radiation events coincidently received by a pair of detectors of the array,
define a line of response based on at least one of longitudinal and circumferential location of the detectors which receive a corresponding coincident pair of events, and
reconstruct the line of responses into an image.
